(19) United States
(12) Patent Application Publication
(10) Pub. No.: US 2026/0096782 A1
(43) Pub. Date: Apr. 9, 2026

(54) Activity Level and Fall Detection Using Accelerometer Data

(71) Applicant: iRhythm Technologies, Inc., San Francisco, CA (US)

(72) Inventors: Yuriko Tamura, San Mateo, CA (US); Elaine Yuiyi Yu, Walnut Creek, CA (US); Jasmine Yu Hu, Shoreline, WA (US); Andrew David Gilbert, Vancouver (CA)

(73) Assignee: iRhythm Technologies, Inc., San Francisco, CA (US)

(21) Appl. No.: 19/347,425

(22) Filed: Oct. 1, 2025

Related U.S. Application Data
(60) Provisional application No. 63/703,820, filed on Oct. 4, 2024.
(60) Provisional application No. 63/703,783, filed on Oct. 4, 2024.
(60) Provisional application No. 63/703,833, filed on Oct. 4, 2024.

(51) Int. Cl. G01C 22/00; A61B 5/00; A61B 5/024; G08B 21/04

(52) U.S. Cl. CPC A61B 5/7264; A61B 5/024; G01C 22/006; G08B 21/043; G08B 21/0446; G08B 21/0453

(57) ABSTRACT

Activity level and fall detection using accelerometer data is described. In one or more implementations, measurements of a user generated by a wearable monitoring device during an observation period are obtained, the measurements including accelerometer data. Physical steps taken by the user are detected based on peaks in the accelerometer data above a threshold, and a step count is generated based on the detected physical steps within predetermined time epochs. Activity level classifications of the user for the predetermined time epochs are generated based on the step count. The step count and the activity level classification for the predetermined time epochs may then be output, such as a notification or in a user interface. Fall predictions may be generated and output by processing the accelerometer data using machine learning models trained to correlate patterns in accelerometer data to fall events.

### RELATED APPLICATIONS

This application claims priority to U.S. Provisional Application No. 63/703,820, filed October 4, 2024, and titled “Activity Level and Step Count Detection,” to U.S. Provisional Application No. 63/703,783, filed October 4, 2024, and titled “Fall Detection Using Chest Accelerometer,” and to U.S. Provisional Application No. 63/703,833, filed October 4, 2024, and titled “Sleep and Activity Prediction with Constrained Conditions,” which are hereby incorporated by reference in their entireties.

### BACKGROUND

Wearable activity trackers and medical monitoring devices have become increasingly prevalent in recent years, offering users the ability to track various aspects of their health and physical activity. These devices typically utilize accelerometers and other sensors to collect data on movement, heart rate, and/or other physiological parameters. However, many existing solutions rely on additional sensors like gyroscopes, which can be power-intensive and costly. Furthermore, conventional approaches often utilize accelerometers sampling at high frequencies (25-100 Hz) and may rely on multiple sensors to calculate body movement and position with multiple possible body placements. These hardware configurations and sampling frequencies limit the ability to achieve longer battery life in wearable devices while maintaining accurate detection capabilities. Additionally, traditional sensor designs face challenges in distinguishing between different types of activities and events that might produce similar accelerometer signatures, which can impact the reliability and accuracy of activity classification and event detection systems.

### BRIEF DESCRIPTION OF THE DRAWINGS

FIG. 1 is a block diagram of a non-limiting example of an environment that is operable to employ activity level and fall detection using accelerometer data as described herein.

FIG. 2 depicts a non-limiting example of a monitoring device.

FIG. 3 depicts a non-limiting system in an example implementation of activity level and fall detection using accelerometer data showing operation of the prediction system of FIG. 1 in more detail.

FIG. 4 shows an example depicting step detection using accelerometer data.

FIG. 5 shows an example of step counting during a structured treadmill activity.

FIG. 6 shows an example of fall detection using chest accelerometer data.

FIGS. 7A and 7B show an example of user interface configurations for displaying health monitoring data and clinical reports.

FIG. 8 shows a flow diagram depicting an algorithm as a step-by-step procedure in an example implementation that is performable by a processing device to generate activity level and step count predictions based on accelerometer measurements.

FIG. 9 depicts a flow diagram depicting an algorithm as a step-by-step procedure in an example implementation that is performable by a processing device to generate fall predictions correlated with cardiac rhythm classifications.

FIG. 10 depicts a flow diagram depicting an algorithm as a step-by-step procedure in an example implementation that is performable by a processing device to generate wellness predictions.

### DETAILED DESCRIPTION

Conventional activity tracking systems typically rely on high-frequency accelerometer sampling rates ranging from 25-100 Hz and often incorporate additional sensors such as gyroscopes to calculate body movement and position across multiple possible body placements. These conventional approaches face several limitations that impact their practical deployment and effectiveness. For example, the high sampling frequencies and reliance on multiple sensors result in substantial power consumption, which limits battery life and reduces the feasibility of extended monitoring periods. Additionally, conventional systems struggle to accurately distinguish between different types of activities and events that produce similar accelerometer signatures, leading to false positives and reduced reliability in activity classification and fall detection. The complexity of multi-sensor configurations also increases device cost and manufacturing complexity while potentially compromising user comfort and compliance during extended wear periods.

Accordingly, techniques, methods, and systems for activity level and fall detection using accelerometer data are described that address these limitations by providing activity level and fall detection using accelerometer data collected at low sampling rates while maintaining high accuracy and enabling comprehensive physiological analysis. In one or more implementations, a wearable monitoring device obtains measurements including accelerometer data during an observation period, with the accelerometer data collected at a sampling rate of less than 5 Hertz (Hz) to conserve battery life and reduce power consumption. Physical steps taken by a user are detected based on peaks in the accelerometer data that exceed a predetermined threshold, enabling accurate step counting even at these lower sampling frequencies. Step counts are generated based on the detected physical steps within predetermined time epochs, and activity level classifications are generated for these epochs based on the step count within each corresponding time epoch. The activity level classification categorizes user activity as sedentary, light, moderate, or vigorous, providing detailed insights into physical activity patterns over extended monitoring periods. In one or more implementations, time-domain and frequency-domain features are extracted from the accelerometer data to enhance the accuracy of activity level classification.

The system further generates fall predictions by processing the accelerometer data using at least one machine learning model specifically trained to correlate patterns in accelerometer data to fall events. The at least one machine learning model is trained using historical accelerometer data and historical outcome data from user populations, enabling accurate differentiation between genuine falls and other activities that might produce similar accelerometer signatures. The system analyzes factors such as impact force, body orientation changes, and post-fall movement patterns to minimize false positives while maintaining high sensitivity to actual fall events.

In implementations where the measurements additionally include electrical potential measurements of a heart of the user, the system generates cardiac rhythm classifications by processing the electrical potential measurements using one or more additional machine learning models trained to correlate patterns in electrical potential measurements to cardiac rhythm classifications. The cardiac rhythm classification identifies specific arrhythmia types such as atrial fibrillation, bradycardia, ventricular arrhythmia, heart block, premature ventricular contractions, supraventricular tachycardia, or normal sinus rhythm. The system correlates fall predictions with concurrent cardiac rhythm classifications and outputs notifications when fall events temporally correlate with detected arrhythmias, providing healthcare providers with comprehensive insights into potential causal relationships between cardiac events and falls.

The described techniques provide substantial advantages over conventional approaches by enabling accurate activity detection and fall prediction using a accelerometer sensor operating at low sampling frequencies to obtain measurements related to user movement and position, thereby extending a battery life of the wearable monitoring device and reducing hardware complexity compared to conventional multi-sensor systems. By way of example, the low-power operation and simplified hardware design make the wearable monitoring device suitable for extended continuous monitoring periods (e.g., two weeks) without charging or battery replacement while maintaining high accuracy in both activity classification and fall detection tasks. Moreover, the integration of accelerometer-based fall detection with concurrent cardiac monitoring provides comprehensive physiological insights that are not achievable through separate analysis of individual sensor modalities. For instance, the correlation between fall events and cardiac irregularities enables healthcare providers to make informed clinical decisions without relying solely on patient recollection, particularly in cases where cardiac events may have contributed to fall incidents. Additionally, the integration of activity level detection with fall detection enables the system to identify correlations between activity patterns and fall events and predict which activity intensities may precipitate falls. Further discussion of these and other examples and advantages are included in the following sections and shown using corresponding figures.

In some aspects, the techniques described herein relate to a method implemented by a processing device, the method including: obtaining measurements of a user generated by a wearable monitoring device during an observation period, the measurements including accelerometer data; detecting physical steps taken by the user based on peaks in the accelerometer data above a threshold; generating a step count based on the detected physical steps within predetermined time epochs; generating an activity level classification of the user for the predetermined time epochs based on the step count within a corresponding predetermined time epoch; and outputting the step count and the activity level classification for the predetermined time epochs.

In some aspects, the techniques described herein relate to a method, wherein the accelerometer data are collected at a sampling rate of less than 5 Hertz.

In some aspects, the techniques described herein relate to a method, wherein the activity level classification includes categorizing an activity of the user during the predetermined time epochs as one of sedentary, light, moderate, or vigorous based on the step count within the predetermined time epochs.

In some aspects, the techniques described herein relate to a method, further including extracting time-domain and frequency-domain features from the accelerometer data, and wherein generating the activity level classification is additionally based on the extracted features.

In some aspects, the techniques described herein relate to a method, further including: generating a fall prediction by processing the accelerometer data using a machine learning model trained to correlate patterns in the accelerometer data to fall events; and outputting the fall prediction.

In some aspects, the techniques described herein relate to a method, further including training the machine learning model to perform the fall prediction using historical accelerometer data and historical outcome data of a user population as training data, wherein the historical accelerometer data is sampled at a same sampling rate as the accelerometer data obtained by the wearable monitoring device.

In some aspects, the techniques described herein relate to a method, wherein the measurements further include electrical potential measurements of a heart of the user, and the method further includes: generating a cardiac rhythm classification by processing the electrical potential measurements using another machine learning model trained to correlate patterns in the electrical potential measurements to cardiac rhythm classifications.

In some aspects, the techniques described herein relate to a method, wherein the cardiac rhythm classification includes an indication of an arrhythmia, and the method further includes: outputting a notification in response to the fall prediction temporally correlating with the arrhythmia.

In some aspects, the techniques described herein relate to a method, wherein the cardiac rhythm classification is one of atrial fibrillation, bradycardia, ventricular arrhythmia, heart block, premature ventricular contraction, supraventricular tachycardia, or normal sinus rhythm.

In some aspects, the techniques described herein relate to a processing device, including: one or more processors; and memory having stored computer-readable instructions that are executable by the one or more processors to perform operations including: obtaining measurements of a user generated by a wearable monitoring device during an observation period, the measurements including accelerometer data; detecting physical steps taken by the user based on peaks in the accelerometer data above a threshold; generating a step count based on the detected physical steps within predetermined time epochs; generating an activity level classification of the user for the predetermined time epochs based on the step count within a corresponding predetermined time epoch; and outputting at least one of the step count or the activity level classification for the predetermined time epochs in a user interface.

In some aspects, the techniques described herein relate to a processing device, wherein the accelerometer data are obtained by an accelerometer of the wearable monitoring device at a sampling rate of less than 5 Hertz.

In some aspects, the techniques described herein relate to a processing device, wherein the activity level classification includes categorizing an activity of the user during the predetermined time epochs as one of sedentary, light, moderate, or vigorous based on the step count within the predetermined time epochs.

In some aspects, the techniques described herein relate to a processing device, wherein the operations further include generating a fall prediction by processing the accelerometer data using a machine learning model trained to correlate patterns in the accelerometer data to fall events.

In some aspects, the techniques described herein relate to a processing device, wherein the measurements further include electrical potential measurements of a heart of the user, and the operations further include: generating a cardiac rhythm classification by processing the electrical potential measurements using another machine learning model trained to correlate patterns in the electrical potential measurements to cardiac rhythm classifications; and outputting the cardiac rhythm classification in the user interface.

In some aspects, the techniques described herein relate to a processing device, wherein the operations further include: correlating the activity level classification with one or both of the fall prediction and the cardiac rhythm classification; and outputting a wellness prediction based on the correlating.

In some aspects, the techniques described herein relate to a system, including: a wearable monitoring device that is wearable by a user to detect one or more measurements of the user during an observation period, the one or more measurements including accelerometer measurements and electrical potential measurements of a heart of the user; and a computing device configured to: receive the one or more measurements from the wearable monitoring device; generate activity level classifications of the user within predetermined time epochs based on the accelerometer measurements within a corresponding predetermined time epoch; generate a fall prediction by processing the accelerometer measurements using a machine learning model trained to correlate patterns in the accelerometer measurements to fall events; and output the activity level classifications and the fall prediction.

In some aspects, the techniques described herein relate to a system, wherein the accelerometer measurements are collected at a sampling rate of less than 5 Hertz.

In some aspects, the techniques described herein relate to a system, wherein the computing device is further configured to generate a cardiac rhythm classification by processing the electrical potential measurements using another machine learning model trained to correlate patterns in the electrical potential measurements to cardiac rhythm classifications.

In some aspects, the techniques described herein relate to a system, wherein the computing device is further configured to: correlate the fall prediction with a concurrent cardiac rhythm classification; and output a notification regarding the fall prediction and the concurrent cardiac rhythm classification.

In some aspects, the techniques described herein relate to a system, wherein to generate the activity level classifications, the computing device is further configured to: detect steps the user has taken based on peaks in the accelerometer measurements that exceed a threshold; generate step counts within the predetermined time epochs based on the detected steps within a given predetermined time epoch; and indicate a given activity level classification for the given predetermined time epoch as one of sedentary, light, moderate, or vigorous based on the step counts within the given predetermined time epoch.

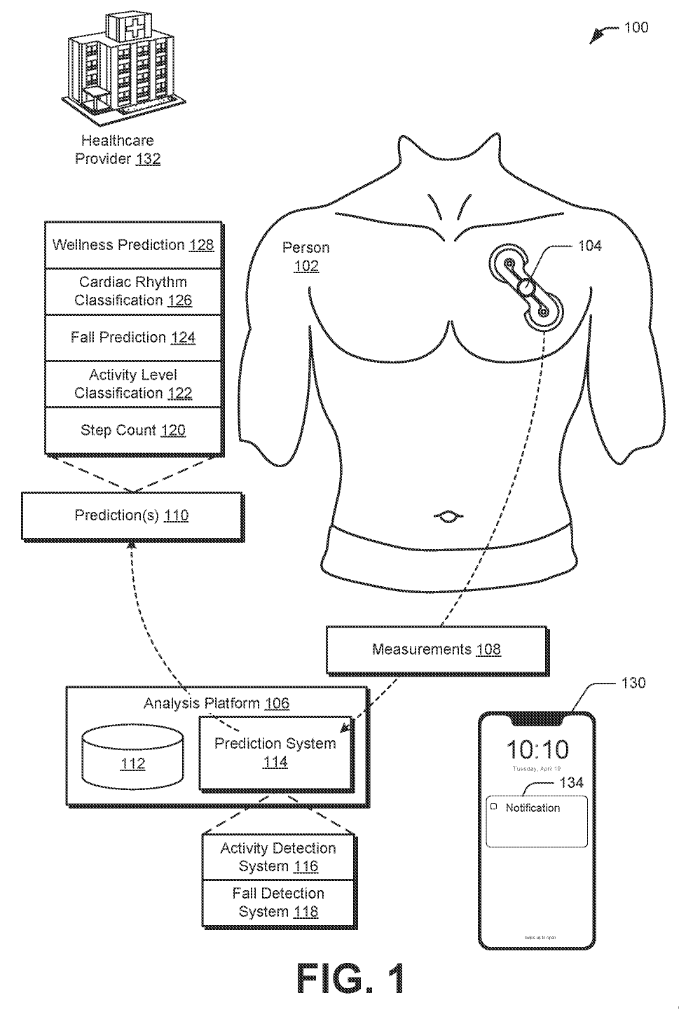

FIG. 1 is a block diagram of a non-limiting example 100 of an environment that is operable to employ activity level and fall detection using accelerometer data as described herein. The illustrated example 100 includes a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102, such as to generate measurements 108. In some scenarios, for instance, the monitoring device 104 may be provided to record electrical activity of the person 102’s heart over an observation period, e.g., lasting some number of seconds or minutes, lasting multiple days, and so on. By way of example, the person 102 may have a magnitude of his or her heart’s electrical potential monitored over time to produce one or more electrocardiograms, which may be used to predict any of a variety of events. In one or more implementations, alternatively or in addition, the monitoring device 104 may be provided to record accelerometer data over the observation period, such as to collect movement and activity data for step counting, activity level classification, and fall detection. In one or more implementations, the accelerometer data may be sampled at a low sampling rate (e.g., in a range between 1-5 Hz, such as 1.6 Hz) to enable longer battery life while maintaining accurate detection capabilities. The monitoring device 104 may output the measurements 108 (e.g., a time sequence of measurements, such as a time sequence of electric potential measurements, acceleration measurements, and/or other types of physical and/or physiological measurements), which may indicate an observation or be used to generate a prediction of one or more events.

As used herein with respect to activity, the term “step” refers to a physical movement. A step may include various types of physical motions or actions, including but not limited to a single footfall during walking or running, a heel strike followed by toe-off during the gait cycle, half of a complete stride cycle involving both left and right foot movements, a climbing motion during stair ascent or descent, a stepping motion during lateral movement or direction changes, a marching step during stationary exercise, a dance step or rhythmic movement pattern, a stepping motion during balance recovery or postural adjustment, and/or any repetitive lower extremity movement that generates characteristic acceleration patterns detectable by the accelerometer sensor.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternatively or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of the observation period has lapsed (e.g., two days) while wearing the monitoring device 104 and/or based on patterns in the aspects of the person 102 being measured.

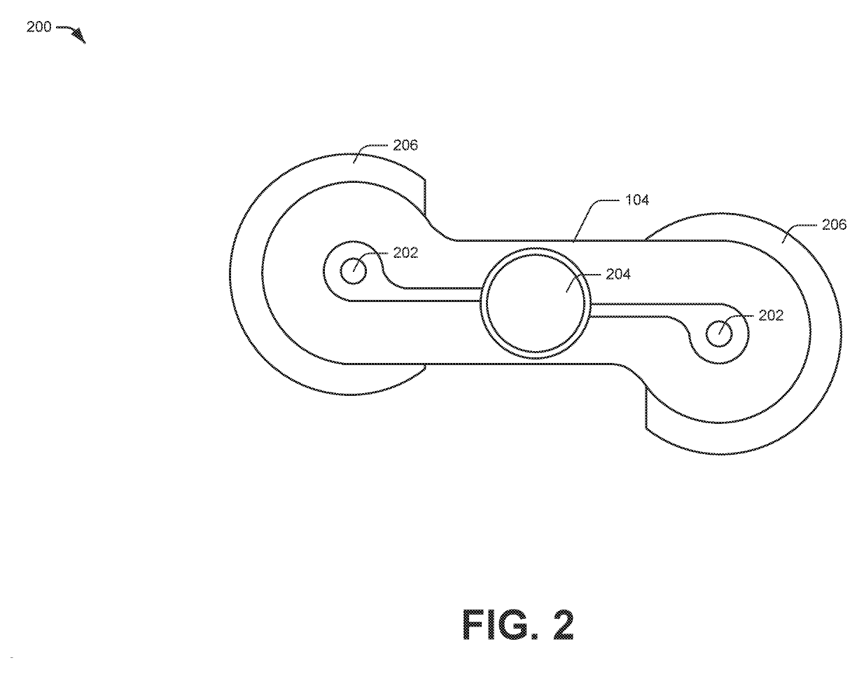

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person 102), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings but may have different functionality, such as functionality that prevents a wearer from viewing the measurements 108.

As used herein, the term “continuous” used in connection with monitoring any signals associated with the person 102 (e.g., acceleration data and/or electrical activity of the person 102’s heart) may refer to an ability of a device to produce measurements substantially continuously, such that the device may be configured to produce the measurements 108 at intervals of time (e.g., every hour, every 30 minutes, every 5 minutes, every minute, every 30 seconds, every second, every half second, and so forth), responsive to an event (e.g., an electrical signal reaching an inflection point such as a peak or a valley), and so forth. The functionality of the monitoring device 104 to produce the measurements 108 along with other measurements and/or to record any of a variety of signals may vary without departing from the spirit or scope of the described techniques.

In one or more implementations, the monitoring device 104 may be configured to offload measurements 108 during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of activity, arrhythmias, fall detection, and so forth. By way of example, the monitoring device 104 may perform light processing of the measurements 108 during wear, such as by averaging or encoding at least a portion of the measurements 108, to reduce the amount of data stored and/or transmitted while preserving information for post-wear analysis. In some such implementations, more comprehensive data processing may occur after the wear period to conserve battery power and reduce memory storage usage.

In one or more implementations, the monitoring device 104 may also implement other power management strategies where data from one type of sensor is used to selectively trigger measurement capture by other sensors. As an illustrative example, additionally or alternatively, accelerometer data indicating potential fall events may trigger enhanced cardiac monitoring data during and immediately following fall episodes, enabling analysis of whether cardiac arrhythmias such as atrial fibrillation, heart block, or ventricular arrhythmias preceded or followed the fall event.

To the extent that the monitoring device 104 may be configured to store the measurements 108 for an entirety of the observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device’s storage.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements 108 are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device’s local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured generate the one or more predictions 110. For example, the measurements 108 may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or the prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104, or the monitoring device 104 may be inserted into an apparatus having a receptacle that interfaces with corresponding contacts of the monitoring device 104. The measurements 108 may be obtained from a storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of the observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc. As noted above, examples of such additional measurements include but are not limited to oxygen saturation (SpO2) measurements. In one or more implementations, the combination of accelerometer data and cardiac measurements may enable enhanced fall detection capabilities, including the ability to correlate fall events with concurrent cardiac arrhythmias such as atrial fibrillation, ventricular arrhythmias, or bradycardia episodes that may contribute to fall risk or be triggered by fall events and/or specific activity levels. In one or more implementations, the analysis performed by the analysis platform 106 may include correlation analysis between accelerometer-detected fall events and concurrent cardiac arrhythmias to identify patterns where specific arrhythmia types may predispose users to falls or where fall events may trigger cardiac irregularities. Additionally or alternatively, the analysis platform 106 may correlate exercise intensity to cardiac events, enabling identification of which activity intensities may precipitate cardiac arrhythmias or other physiological responses. The activity intensity may provide context to detected falls that supports insights not achievable with separate analyses, such as determining which exercise intensities are likely to cause falls for particular users or patient populations.

In one or more implementations, the analysis platform 106 may be implemented in whole or in part at the monitoring device 104. Alternatively or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes a storage device 112. In accordance with the described techniques, the storage device 112 may be configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In one or more implementations, the storage device 112 may also store activity thresholds, step detection parameters, fall detection algorithms, algorithms for activity classification, historical activity patterns, baseline accelerometer data for comparison purposes, body angle calculation parameters, and multi-modal data fusion algorithms that determine how to combine accelerometer data with other physiological measurements such as ECG or SpO2 data for enhanced prediction accuracy. One or more algorithms may be or may include machine learning algorithms. By way of example, at least one machine learning algorithm may be trained to recognize temporal relationships between cardiac events and subsequent falls. In one or more implementations, the storage device 112 may also maintain arrhythmia classification models, fall-arrhythmia correlation databases, cardiac event timing and activity timing relative to fall occurrences, and/or algorithms for identifying causal relationships between specific arrhythmia types and fall risk factors.

In the illustrated example 100, the analysis platform 106 also includes the prediction system 114. The prediction system 114 represents functionality to process the measurements 108 to generate the one or more prediction(s) 110. Alternatively or in addition, the prediction system 114 may output one or more time sequences indicating an observation or prediction of one or more events over time. It is also to be appreciated that the prediction system 114 may output different combinations of multiple predictions in variations.

In at least one implementation, the prediction system 114 uses machine learning to generate at least a portion of the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively or additionally, the prediction system 114 may include logic (a machine learning model and/or other types of logic) to preprocess the measurements 108, such as to extract various cardiovascular, movement, and/or other features from the sequences of measurements. The one or more predictions 110 may be the output of the prediction system 114, for example.

In various examples, the prediction system 114 may be representative of and/or may include an activity detection system 116 and a fall detection system 118, and the one or more predictions 110 may include and/or may be representative of a step count 120, an activity level classification 122, a fall prediction 124, a cardiac rhythm classification 126, and/or a wellness prediction 128. For instance, as further described in more detail below, the activity detection system 116 may be operable to implement accelerometer-based techniques to generate the step count 120 and the activity level classification 122. The activity detection system 116 may receive accelerometer data and detect peaks above a threshold to identify steps as part of the step count 120, count steps within predetermined epochs, and classify activity intensity based on the step count. The activity level classification 122, for instance, may categorize activity levels as sedentary, light, moderate, or vigorous based on step counts within the predetermined epochs and extracted features from accelerometer data. The activity detection system 116 can dynamically adjust thresholds for improved step detection accuracy and may incorporate time-domain and frequency-domain features for enhanced activity classification.

The fall detection system 118 may be operable to analyze accelerometer data patterns to detect fall events and correlate them with cardiac data to generate the fall prediction 124, which may indicate the number and timing of falls, including their potential correlation with arrhythmias. In one or more implementations, the fall detection system 118 may leverage accelerometer data of the measurements 108 to identify fall events and correlate them with cardiac data of the measurements 108, providing context for arrhythmia diagnosis and patient care. The fall detection system 118 may employ one or more algorithms (e.g., machine learning algorithms and/or conventionally programmed algorithms) to analyze the accelerometer data, differentiating between genuine falls and other activities by considering factors such as impact force, body orientation changes, and post-fall movement patterns. The fall detection system 118 may further employ one or more algorithms (e.g., machine learning algorithms and/or conventionally programmed algorithms) to analyze cardiac data of the measurements 108 (e.g., ECG waveform data) to classify a cardiac rhythm, resulting in the cardiac rhythm classification 126.

By way of example, the fall detection system 118 may be configured to identify a specific arrhythmia type preceding a event. By way of example, the cardiac rhythm classification 126 may indicate bradycardia episodes that may cause dizziness, atrial fibrillation episodes that may affect cardiac output and balance, and/or ventricular arrhythmias that may cause sudden weakness or syncope leading to falls. The fall detection system 118 may also analyze whether fall events themselves trigger subsequent cardiac arrhythmias due to the physical stress or emotional response associated with falling and/or a correlation between activity levels (e.g., as detected by the activity detection system 116) and fall events. The fall detection system 118 may further correlate exercise intensity with fall events and/or cardiac arrhythmias to determine which activity intensities are most likely to precipitate falls, providing insights into activity-related fall risk patterns that are not achievable through separate analysis of fall detection and activity monitoring. By integrating fall detection with simultaneous cardiac data analysis, the fall detection system 118 may uncover relationships between falls and cardiac events, offering healthcare providers a comprehensive tool for assessing patient health and fall risks.

The wellness prediction 128 may provide comprehensive physiological insights that combine movement analysis with cardiac context, enabling detection of relationships between activity patterns, cardiac wellness metrics, and overall health status. The wellness prediction 128 may include correlations between daily activity levels and cardiac health indicators that indicate patterns suggesting cardiovascular fitness, exercise tolerance, or potential health risks. For instance, the wellness prediction 128 may indicate relationships between step counts and heart rate variability, correlations between activity intensity and cardiac arrhythmia frequency, or associations between movement patterns and overall cardiovascular wellness. The wellness prediction 128 may also provide insights into how different activity levels affect cardiac function over time, including information about exercise capacity, recovery patterns, and the impact of physical activity on heart health. By integrating accelerometer-derived activity data with concurrent cardiac measurements, the wellness prediction 128 may identify optimal activity levels for individual users, indicate early signs of cardiovascular decline, and/or provide personalized recommendations for maintaining or improving overall health.

The wellness prediction 128 may further include relationships between fall events and overall health patterns, correlations between fall frequency and cardiovascular wellness, associations between fall risk and activity levels, and how fall data combined with activity and cardiac measurements provide comprehensive health insights for healthcare providers. The wellness prediction 128 may further include comprehensive wellness scores that combine multiple physiological parameters, enabling healthcare providers to assess patient health holistically rather than through isolated measurements. This multi-modal approach may provide clinical benefits by supporting preventive care strategies and enabling early intervention when certain patterns are detected in the combined activity and cardiac data.

Further illustrated in the example 100 is an accessory device 130 and a healthcare provider 132. The accessory device 130, for instance, may include one or more devices associated with the person 102 and/or the monitoring device 104, such as those described above. For instance, the accessory device 130 may include a display device (e.g., a smartphone and/or a personal display device) to display the one or more predictions 110 and/or to control functionality of the monitoring device 104. The accessory device 130 may also display a notification 134, as will be further elaborated below.

The healthcare provider 132, for instance, may be representative of one or more additional processing devices associated with an authorized medical system, e.g., practitioner devices, electronic health record systems, diagnostic imaging equipment, laboratory information systems, telemedicine platforms, clinical decision support tools, and so forth. In various implementations, the notification 134 may be generated based on the one or more predictions 110, such as alerts for detected fall events, changes in activity levels, changes in the wellness prediction 128, and/or other detected events, and may be displayed by the accessory device 130 to communicate information to the person 102, healthcare providers, caregivers, and/or emergency contacts. As a non-limiting example, the prediction system 114 may generate reports and/or alerts based on the fall prediction 124, which may lead to more targeted interventions and improved fall prevention strategies.

In various examples, one or more operations of the analysis platform 106, the prediction system 114, the activity detection system 116, and/or the fall detection system 118 are performable by one or more of the monitoring device 104, the accessory device 130, the devices and systems of the healthcare provider 132, and/or one or more additional devices not shown.

FIG. 2 depicts a non-limiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102’s heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the person 102’s heart’s electrical activity. In one or more implementations, the processor further generates one or more communicable packages of data that include one or more of the measurements 108 and/or other measurements, such oxygen saturation (SpO2) measurements. Alternatively or additionally, the processor produces and/or causes storage of other data, which may be used for predicting classifications of physiological conditions, e.g., arrhythmias, activity levels, and the like.

In implementations where the monitoring device 104 is configured for wireless transmission, the transmitter 204 may transmit the measurements 108 wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternatively or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Activity Level and Fall Detection Using Accelerometer Data

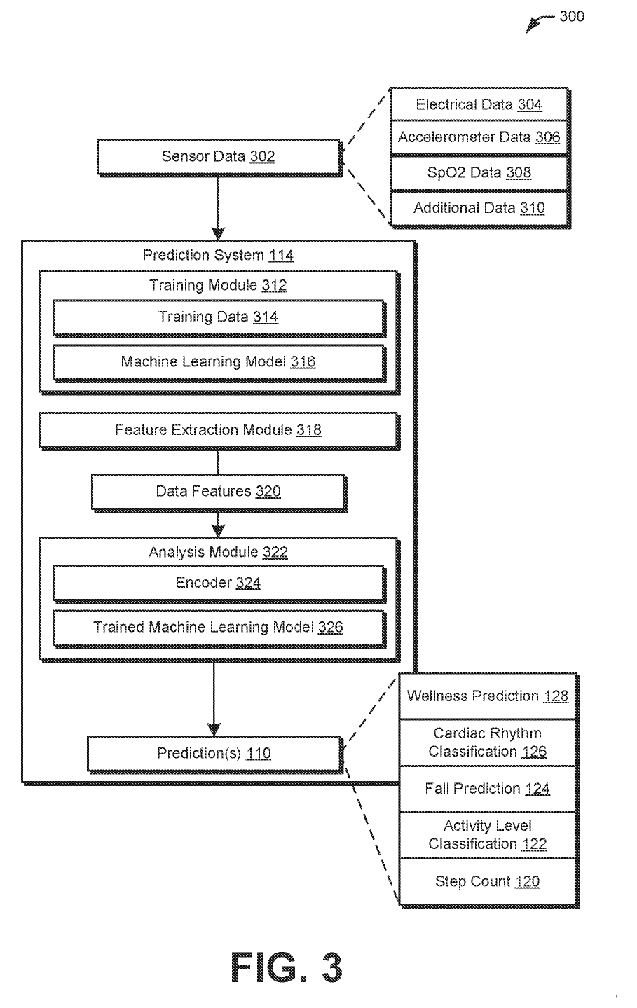

FIG. 3 depicts a non-limiting system in an example implementation 300 of activity level and fall detection using accelerometer data showing operation of the prediction system 114 of FIG. 1 in more detail.

To begin in this example, the prediction system 114 receives sensor data 302, which may include electrical data 304 (e.g., electrical potential measurements and/or ECG data), accelerometer data 306, SpO2 data 308 (e.g., oxygen saturation data), and/or various additional data 310. In various examples, the sensor data 302 is collected by one or more devices and/or sensors, such as the wearable monitoring device 104. The sensor data 302 can include time-sequenced instances of data, such as continuous data, data collected at predetermined intervals (e.g., per half-second interval, per minute interval, per five minute interval, etc.) for the length of an observation period, e.g., a single day, multiple days during a week, for a month, and so forth. The sensor data 302 may be the measurements 108 introduced with respect to FIG. 1, for example. In one or more implementations, the accelerometer data 306 is collected at a sampling rate that is less than 5 Hz (e.g., a 1.6 Hz sampling rate). Generally, the sensor data 302 is processed by the prediction system 114 to generate the one or more predictions 110, which may include one or more of the step count 120, the activity level classification 122, the fall prediction 124, the cardiac rhythm classification 126, and the wellness prediction 128, in accordance with the techniques described in more detail below.

For instance, the prediction system 114 includes a training module 312 that is operable to train a machine learning model 316 using training data 314 to perform one or more activity detection and fall detection tasks. The activity detection and fall detection tasks, for instance, involve generation of the one or more predictions 110, such as prediction of step counts, activity levels, and fall events based on patterns in the accelerometer data 306; correlating activity patterns with cardiac data from the electrical data 304 for the wellness prediction 128; monitoring body position based on the electrical data 304 and heart data based on the accelerometer data 306 for fall recovery analysis; and detecting fall events based on patterns in the accelerometer data 306, which may be correlated with arrhythmias based on the electrical data 304. These tasks may include the classification of activity levels as sedentary, light, moderate, or vigorous; counting of steps within predetermined epochs; detection of fall events; and correlation of falls with cardiac data (e.g., based on the electrical data 304). In one or more implementations, the tasks may also include identification of cardiac arrhythmias from the electrical data 304 that may precede or follow fall events, enabling analysis of causal relationships between specific arrhythmia types and fall occurrences. Accordingly, the machine learning model 316 is trained to correlate patterns in the sensor data 302, such as various electrical potential measurements of the electrical data 304 and/or accelerometer measurements and body angle calculations of the accelerometer data 306, to the one or more predictions 110. It is to be appreciated that more than one machine learning model 316 may be separately trained, such as separate machine learning models 316 for the different one or more predictions 110.

The previous examples describe various instances of artificial intelligence (“AI”) models and/or machine learning models. In one or more examples, an AI model, e.g., a machine learning model, refers to a computer representation that is tunable (e.g., through training and retraining) based on inputs without being actively programmed by a user to approximate unknown functions, automatically and without user intervention. For instance, the term “machine learning model” includes a model that utilizes algorithms to learn from, and make predictions on, known data by analyzing training data to learn and relearn to generate outputs that reflect patterns and attributes of the training data. In the context of activity level and fall detection using accelerometer data, machine learning models are implementable (e.g., by one or more processing devices of the prediction system 114) to analyze accelerometer data patterns and correlate them with cardiac data to identify activity levels, detect fall events, and generate comprehensive physiological insights.

The training module 312 is further operable to initialize various parameters of the machine learning model 316, which are usable by the machine learning model 316 as internal variables to represent and process information during training. These parameters are further usable to represent inferences gained through training. In one or more implementations, the training data 314 are separated into batches to improve processing and optimization efficiency of the parameters of the machine learning model 316 during training, which is particularly beneficial for model accuracy when processing large volumes of accelerometer time-series data collected at low sampling rates and correlating movement patterns with concurrent cardiac measurements.

In the present example, the training data 314 may include historical electrical potential measurements (e.g., such as ECG) and accelerometer measurements data from a population of users along with corresponding historical outcome data, such as arrhythmia classifications, activity classifications, step counts, fall events, and/or overall wellness classifications. These data may be collected from clinical studies, activity monitoring studies, or other sources where the electrical data 304 and the accelerometer data 306 are recorded simultaneously. In some cases, the training data 314 may also include additional physiological measurements such as oxygen saturation levels (e.g., the SpO2 data 308), and/or additional relevant biomarkers. The training data 314 may be labeled with various activity and fall-related information, such as the presence or absence of activity periods, types of activities (e.g., walking, running, sleeping), step counts, fall events, body positions, and so forth.

The training data 314 may also be labeled with various heart rhythm-related information. That is, in order to train the machine learning model for activity level and fall detection, the training data 314 may provide examples of “what is to be learned” by the machine learning model, e.g., as a basis to learn patterns from the data. For activity and fall detection applications, the training data 314 may include labeled datasets of accelerometer measurements and cardiac data from users with known activity patterns, fall events, and cardiac conditions, as well as measurements from individuals with normal activity levels and no fall history. The training module 312, for instance, may collect and preprocess the training data 314 that includes input features (e.g., accelerometer waveforms, body angle calculations, step patterns, ECG data, heart rate patterns) and corresponding target labels (e.g., “sedentary activity,” “vigorous activity,” “fall event detected,” “fall correlated with arrhythmia,” or specific activity and fall classifications). The training data 314 may further be labeled with physiological features such as normal sinus rhythm, atrial fibrillation episodes, ventricular arrhythmias, bradycardia events, heart block occurrences, premature ventricular contractions, supraventricular tachycardia, and/or other cardiac rhythm abnormalities that may be temporally associated with fall events or activity level changes.

The training data 314 may be received as an input by the machine learning model 316 and used as a basis for generating predictions based on a current state of parameters of layers and corresponding nodes of the model, a result of which is output as output data, e.g., activity level classifications, step counts, fall event detections, correlations between falls and cardiac events, etc.

A machine learning model, for instance, may be configurable using a plurality of layers having, respectively, a plurality of nodes. The plurality of layers is configurable to include an input layer, an output layer, and one or more hidden layers. In the context of activity level and fall detection, the input layer may receive the sensor data 302 or features thereof, including magnitude calculations, body angle measurements, step detection peaks, movement patterns, activity level indicators, and cardiac features. The hidden layers, for instance, process these inputs through weighted connections to identify complex patterns indicative of activity levels, fall events, and correlations between movement and cardiac status, e.g., patterns that are not detectable using conventional analysis modalities that rely solely on basic activity tracking. The output layer may produce the one or more predictions 110, including the step count 120, the activity level classification 122, the fall prediction 124, and/or the wellness prediction 128, as well as correlations between fall events and cardiac arrhythmias. Calculations are performed by the nodes within the layers via hidden states through a system of weighted connections that are “learned” during training of the machine learning model 316 to implement a variety of activity detection and fall detection tasks.

In some implementations, different training schemes and/or model architectures are employed based on what the one or more predictions 110 are to include. For instance, a composition and structure of the training data 314 may vary depending on the specific type of prediction to be generated. In an example in which the one or more predictions 110 are to indicate a binary classification of activity presence (e.g., whether a user is active or inactive), the training data 314 may be labeled with yes/no indicators. In an additional or alternative example in which the one or more predictions 110 are to include granular predictions such as specific activity levels, step counts, and/or fall severity, the training data 314 includes detailed annotations that pertain to the granular predictions. In an example in which the one or more predictions 110 are to indicate correlations between activity patterns from the accelerometer data 306 and cardiac wellness metrics from the electrical data 304, the training data 314 may include wellness insights associated with various activity levels and cardiac measurements. In an additional or alternative example in which the one or more predictions 110 are to include comprehensive analyses such as fall recovery monitoring using body position and heart data, cardiac-activity wellness correlations, and fall event contextualization with arrhythmia data from the electrical data 304, the training data 314 may include multi-modal input data in order to capture the complex relationships between movement, cardiac status, and physiological outcomes.

In some examples, the training data 314 are structured to support multi-task learning, where the machine learning model 316 can simultaneously predict multiple aspects of activity level and/or fall detection, such as activity level and fall risk in combination, as well as activity-cardiac correlations and fall detection with physiological context, such as wellness insights from activity-heart rate relationships and fall recovery patterns from body position-cardiac data combinations. In additional or alternative examples, the training module 312 trains the machine learning model 316 on a per task basis, such as to implement a first round of training to train the machine learning model 316 to perform activity-cardiac correlation analysis and a second round of training to train the machine learning model 316 to perform fall detection with cardiac contextualization. In this way, the techniques described herein support targeted training of the machine learning model 316 for particular tasks, which improves model performance and efficiency to perform discrete aspects of activity and fall detection as well as complex multi-modal physiological insights that combine the accelerometer data 306 with the electrical data 304 rather than basic activity classification alone.

In one or more implementations, the training module 312 trains the machine learning model 316 using an iterative process of adjusting weights and learning parameters to minimize a loss function. For example, the training module 312 may use backpropagation and/or gradient descent algorithms to update parameters of the model based on a difference between predicted and actual activity and fall classifications in the training data 314. A learning rate, batch size, and/or number of epochs may be tuned to optimize the performance of the machine learning model 316.

Training of the machine learning model 316 can include calculation of a loss function to quantify a loss associated with operations performed by nodes of the machine learning model 316. The loss function is configurable in various ways to control operation and/or functionality of the machine learning model 316. For instance, the loss function may be designed to prioritize accuracy in detection of fall events while minimizing false positives, and to optimize correlation accuracy between activity patterns and cardiac wellness metrics. Calculation of the loss function, for instance, includes comparing a difference between predictions specified in the output data (e.g., predicted activity levels, fall events, or cardiac-activity correlations) with target labels specified by the training data 314 (e.g., clinically confirmed activity classifications and documented fall events). The loss function is configurable in a variety of ways, examples of which include a quadratic loss function as part of a least squares technique for continuous activity parameters, cross-entropy loss for classification tasks such as activity level categorization, custom loss functions that incorporate clinical risk factors specific to fall detection and cardiac-activity correlations, and so forth.

Furthermore, a variety of architectures/types of the machine learning model 316 are considered. In one or more implementations, the machine learning model 316 may include a neural network, such as a convolutional neural network (CNN), recurrent neural network (RNN), or a combination thereof. In some instances, the machine learning model 316 incorporates one or more U-Net and/or ResNet architectures, features, or components. The model may also be implemented as an ensemble of different algorithms that combines one or more decision trees, random forests, and/or gradient boosting machines with neural network approaches. By way of example, CNNs may be used for analyzing accelerometer waveform patterns and detecting fall signatures. As another example, long short-term memory (LSTM) neural networks may be used to analyze temporal movement patterns and correlate activity sequences with cardiac events. In still other examples, generative adversarial networks (GANs), decision trees (e.g., for activity level classification and fall risk assessment), support vector machines, linear regression, logistic regression for binary fall detection, Bayesian networks, random forest learning for feature importance in accelerometer and cardiac data correlation, dimensionality reduction algorithms, boosting algorithms, deep learning neural networks, and so forth may be employed. It is to be appreciated that the above examples are given by way of illustration, and other configurations may be used without departing from the spirit or scope of the described techniques.

In some examples, techniques such as dropout or regularization may be employed by the training module 312, such as to prevent overfitting. The training process may continue until the model achieves a desired level of accuracy on a validation dataset and/or until a predetermined number of iterations have been completed. This approach allows the machine learning model 316 to learn complex patterns in the sensor data 302 that are indicative of relationships between activity patterns from the accelerometer data 306 and cardiac wellness metrics from the electrical data 304, correlations between body position changes and heart rate responses during fall recovery, temporal associations between arrhythmias and fall events, and multi-modal insights that combine movement and cardiac data to identify subtle features and develop physiological understanding that is not possible using conventional analysis methods. By way of example, it may be impractical or impossible to manually analyze the sensor data 302 to arrive at such insights. This is by way of example and not limitation, and a variety of suitable training techniques are considered.

Multiple attention heads, for instance, may allow the machine learning model 316 to allocate resources to focus on different aspects of the input data to make distinct predictions. Continuing with the above example in which the predictions 110 indicate activity-cardiac correlations, each analysis head may be trained to detect specific relationships between movement patterns from the accelerometer data 306 and physiological responses from the electrical data 304, which enables the prediction system 114 to provide comprehensive multi-modal analysis of the sensor data 302 for wellness insights and fall recovery monitoring. In this way, the techniques described herein support adaptability of the prediction system 114 to efficiently provide focused activity-cardiac correlation information and fall detection with physiological context in addition to or as an alternative to the step count 120, the activity level prediction122, and the fall prediction 124.

Once the machine learning model 316 is trained, the sensor data 302 is processed by a feature extraction module 318, which generates data features 320. For instance, the feature extraction module 318 preprocesses the sensor data 302 to generate usable (e.g., processable) inputs for the trained machine learning model 326. The feature extraction module 318 may generate at least a portion of the data features 320 based on various properties of the accelerometer signal present in the accelerometer data 306, such as magnitude calculations, body angle measurements, step detection peaks, movement patterns, and/or activity level indicators. These extracted features can include time-domain, frequency-domain, and statistical measures that capture relevant information about movement and body position. It is to be appreciated that the training data 314 may include accelerometer data obtained at the same sampling frequency as the accelerometer data 306, at least in some implementations.

The feature extraction module 318 may be further operable to perform body angle calculations as a part of outputting the data features 320. In some implementations, body angle techniques may be used to extract positional information from the accelerometer data 306 to determine body position changes. The feature extraction module 318 may analyze variations in accelerometer orientation, such as changes in gravitational vector components, which can be correlated to and/or influenced by body position and movement activity. These position-related properties of the accelerometer data 306 may be used to derive body angle measurements and activity patterns. The body angle-derived features may also be combined with other data features 320 to provide a comprehensive set of inputs for the trained machine learning model 326. In this way, the prediction system 114 is able to capture both movement and positional information from a single accelerometer signal, which improves detection of correlation between body position and activity events and is not possible using conventional modalities.

In one or more implementations, the feature extraction module 318 may also extract cardiac features from the electrical data 304, such as heart rate variability, QRS complex morphology, R-R interval patterns, and arrhythmia signatures that may be correlated with fall risk or activity level changes.

The feature extraction module 318 may further implement a variety of additional techniques such as wavelet decomposition, principal component analysis, peak detection, statistical analysis, and/or other signal processing methods to isolate and quantify relevant aspects of the electrical data 304 and/or the accelerometer data 306. For example, step detection algorithms may be applied to identify peaks above predetermined thresholds, while magnitude calculations may provide information about overall activity levels. The feature extraction module 318 may incorporate both time-domain features (such as mean, variance, and peak characteristics) and frequency-domain features (such as spectral energy and dominant frequencies) for enhanced activity classification. The feature extraction module 318 may further optimize and/or refine the data features 320, such as based on a discriminative ability of the data features 320 to detect particular activity or fall events.

Once extracted, an analysis module 322 configures the data features 320 for input to a trained machine learning model 326 (e.g., the machine learning model 316 once output by the training module 312). In one or more implementation, the analysis module 322 uses an encoder 324. The encoder 324 is configurable to process and compress input data into a compact representation and can include one or more of a convolutional encoder, recurrent encoder, transformer encoder, one or more autoencoder variants, and so forth. The encoder 324, for instance, generates compressed representations from the data features 320 that can be efficiently processed by the trained machine learning model 326. In an example, the encoder 324 reduces a dimensionality of the data features 320 while preserving relevant information, creating a compact representation that serves as a suitable input to the trained machine learning model 326.

The analysis module 322 generates the one or more predictions 110 for output by processing the encoded data features 320 using the trained machine learning model 326. The predictions 110 may include a variety of information. By way of example, the step count 120 may indicate a number of steps taken, the activity level classification 122 may indicate different levels of physical activity, the fall prediction 124 may indicate detection of fall events in relation to cardiac activity and/or physical activity, and the wellness prediction 128 may provide comprehensive physiological insights that combine movement analysis with cardiac context and/or additional predictions that provide supplementary analysis results. In one or more examples, the one or more predictions 110 may include a confidence interval, e.g., a confidence in the associated value or condition.

In at least one implementation, the step count 120 may include a total number of steps detected within predetermined epochs during the observation period. The step count 120 may be generated by detecting peaks in the accelerometer data 306 above a threshold to identify individual steps, counting the detected steps within predetermined time epochs (e.g., per minute, per hour, or per day), and providing accurate step counting while operating at lower sampling frequencies compared to conventional systems. The activity detection system 116 may dynamically adjust the threshold based on user-specific patterns or environmental conditions to improve step detection accuracy. The activity level classification 122 may distinguish between levels of activity including but not limited to sedentary, light activity, moderate activity, vigorous activity, or combinations thereof. The activity level classification 122 may classify activity intensity based on the step count within predetermined epochs, incorporating extracted features from the accelerometer data 306 such as time-domain and frequency-domain characteristics. As a non-limiting example, movement equivalent to walking speed of 2 miles per hour (mph) or greater may indicate light activity, whereas speeds of less than 2 mph may indicate inactivity/rest. The fall prediction 124, for instance, may indicate whether a user associated with the sensor data 302 has experienced no falls, a risk of experiencing future falls (e.g., low fall risk, moderate fall risk, high fall risk, etc.), a timing of experienced falls, and any correlation with cardiac arrhythmias during detected falls, just to name a few.

For instance, the fall prediction 124 may include an indication of a correlation between a fall event and an additional physiological event, such as a cardiac event, activity event, and so forth, such as when the trained machine learning model 326 is operable to identify and analyze relationships between fall occurrences and various cardiac activity or physical activity levels. For example, the fall prediction 124 may indicate that fall events are more likely to occur during periods of increased activity for a particular user. Combining the fall prediction 124 with the cardiac rhythm classification 126 may indicate temporal associations between fall events and specific cardiac arrhythmias, such as episodes of atrial fibrillation, bradycardia events that may cause dizziness or syncope, or ventricular arrhythmias that may result in sudden weakness leading to falls. Such insights may offer a comprehensive view of physiological responses to and causes of fall events, enabling healthcare providers to better understand whether falls are primarily mechanical in nature or potentially precipitated by underlying cardiac conditions. Moreover, the fall prediction 124 and cardiac rhythm classification 126 may supply clinicians with detailed information to make informed decisions without relying solely on patient recollection.

Additionally, the accelerometer data 306 may be used to detect body position changes during daily activities, which can influence occurrence and/or severity of fall events. By incorporating such positional information, the trained machine learning model 326 may generate accurate fall predictions 124 that take into account a relationship between body position and fall risk. For instance, the trained machine learning model 326 is able to process the accelerometer data 306 to generate a fall prediction 124 that indicates a correlation between user behaviors, e.g., a body position during activities, and fall events. In various implementations, the trained machine learning model 326 may also analyze the electrical data 304 to generate a cardiac rhythm classification 126 that identifies cardiac arrhythmias that commonly occur in specific body positions or during particular activity levels, enabling prediction of fall risk based on both movement patterns and concurrent cardiac status.

In various examples, the sensor data 302 further include the SpO2 data 308 and/or the various additional data 310. These additional measurements may be input to the machine learning model 316 along with the electrical data 304 and/or the accelerometer data 306 to predict the one or more predictions 110. Accordingly, the techniques described herein support multi-modality predictions that provide insights not capable using conventional techniques that rely solely on the accelerometer data 306 or solely on the electrical data 304.

In some implementations, the SpO2 data 308 may be utilized to enhance the accuracy of and/or validate the activity and fall predictions. For instance, the analysis module 322 may process the SpO2 data 308 in conjunction with the electrical data 304 and/or the accelerometer data 306 to identify potential activity periods or fall events. The trained machine learning model 326 may be configured to detect changes in oxygen saturation levels during activity periods, which may coincide with increased physical exertion, and correlate these changes with movement patterns in the accelerometer signal. Additionally or alternatively, the trained machine learning model 326 may be configured to detect changes in oxygen saturation levels and correlate these changes with arrhythmias determined from the electrical data 304. By combining these data sources, the prediction system 114 is operable to distinguish between different types of activity and fall events with enhanced accuracy.

By way of example, different types of activity events may be characterized by distinct accelerometer patterns, such as walking showing regular periodic peaks while running shows higher frequency and amplitude variations. Similarly, the cardiac rhythm classification 126 may identify different types of cardiac arrhythmias that may be associated with distinct fall patterns, such as bradycardia-related falls showing gradual onset versus sudden collapse patterns associated with ventricular arrhythmias. This multi-modal approach enables nuanced and accurate activity and fall predictions, which reduces incidence of false positives and provides additional context for activity levels and fall risk detected, including the ability to distinguish between mechanically caused falls and those potentially precipitated by underlying cardiac conditions.

The following discussion describes techniques that are implementable utilizing the previously described systems and devices. In portions of the following discussion, reference will be made to FIGS. 1-3.

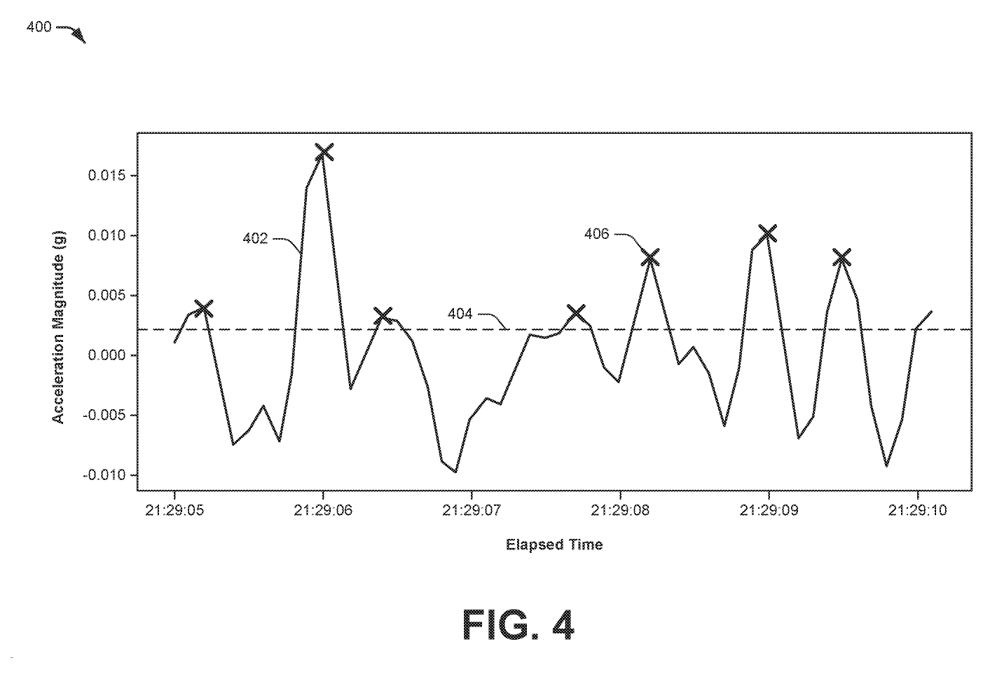

FIG. 4 shows an example 400 depicting step detection using accelerometer data. The example 400 includes an acceleration magnitude 402 plotted over elapsed time, where the acceleration magnitude 402 represents the total magnitude of acceleration forces detected by the accelerometer (e.g., the sensors 202) of the monitoring device 104. In one or more implementations, the acceleration magnitude 402 may be calculated as the vector sum of acceleration components in three orthogonal directions (x, y, and z axes). The acceleration magnitude 402 may be a processed signal where the effect of gravity is removed, for example. The acceleration magnitude 402 depicts periodic variations that correspond to the cyclical nature of human walking patterns, with distinct peaks occurring at regular intervals that align with individual step events.

The example 400 includes a threshold 404 for identifying the individual step events based on the acceleration magnitude 402. In one or more implementations, the prediction system 114 may dynamically adjust the threshold 404 based on variations observed in the acceleration magnitude 402 to improve step counting accuracy across different walking speeds, terrains, or user movement patterns. The dynamic threshold may allow the prediction system 114 to adapt to individual user characteristics and/or environmental conditions that may affect the amplitude of acceleration signals during walking. In at least one variation, however, the threshold 404 is a static threshold.

Multiple step peaks 406 are visible in the acceleration magnitude 402, where each step peak 406 corresponds to a local maximum where the acceleration magnitude 402 exceeds the threshold 404, e.g., due to foot impact or body movement during the walking cycle. The step peaks 406 correspond to step events as detected by the activity detection system 116. The activity detection system 116 may generate the step count 120 by counting the number of peaks that exceed the threshold 404 within predetermined time epochs. The analysis platform 106 may apply additional filtering or validation algorithms to the detected step peaks 406 to eliminate false positives caused by non-walking activities or sensor noise, at least in one or more implementations.

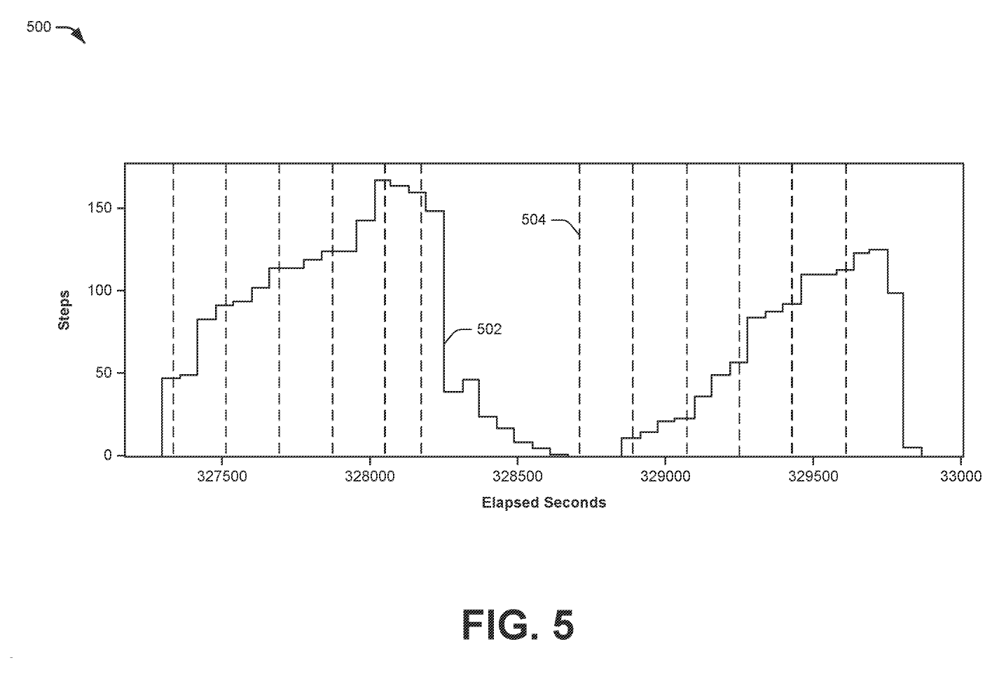

FIG. 5 shows an example 500 of step counting during a structured treadmill activity. The example 500 includes a step count plot 502 that includes step measurements over time (e.g., in elapsed seconds), providing a visual representation of how the step count 120 may be organized and analyzed for activity classification purposes. The step count plot 502 includes multiple vertical dashed lines representing stage boundaries 504, with each stage boundary 504 representing a predetermined time interval during which the treadmill is kept at a pre-determined speed and incline. By way of example, the example 500 may be a cardiac stress test that follows the Bruce Protocol.

The step count plot 502 shows the step counts in measurement bins during which the step count 120 is summed. The measurement bins may have a length of seconds (e.g., 5 seconds, 10 seconds, 30 seconds), minutes (e.g., one minute, five minutes), or another predetermined duration that facilitates the step count 120 analysis based on the sampling rate of the accelerometer. In one or more implementations, the measurement bins may be or may be further grouped into epochs for activity level (e.g., intensity) classification, allowing the analysis platform 106 to process step count data in discrete segments rather than as a continuous stream. In one or more implementations, the prediction system 114 may analyze the step count 120 within each epoch to generate the activity level classification 122 in order to categorize physical activity into distinct intensity levels. By way of example, the activity detection system 116 may classify the activity as sedentary when the step count 120 within an epoch fall below a first, lowest activity threshold, indicating minimal physical movement. The activity detection system 116 may classify the activity as light activity when the step count 120 within the epoch exceeds the first activity threshold but remains below a second activity threshold, suggesting light movement such as slow walking or basic daily activities. The activity detection system 116 may classify the activity as moderate activity when the step count 120 within the epoch is greater than or equal to the second activity threshold but less than a third, highest activity threshold, indicating activities such as brisk walking or light exercise. The activity detection system 116 may indicate vigorous activity when the step count 120 is greater than or equal to the third activity threshold within the epoch, suggesting high-intensity activities such as running or intense physical exercise.

In one or more implementations, such as described with respect to FIG. 3, the feature extraction module 318 may extract both time-domain and frequency-domain features from the accelerometer data 306 to enhance activity intensity classification accuracy. By way of example, the time-domain features may include statistical measures such as mean, variance, and standard deviation of acceleration magnitude within each of the epochs, providing information about movement patterns and intensity variations. The frequency-domain features may be derived through spectral analysis of the accelerometer data 306, which may provide periodic patterns and frequency characteristics that correspond to different types of physical activities. The analysis module 322 may combine the step count 120 with these extracted features to generate more accurate activity level classifications 122 that account for both movement quantity and movement quality characteristics within each of the epochs.

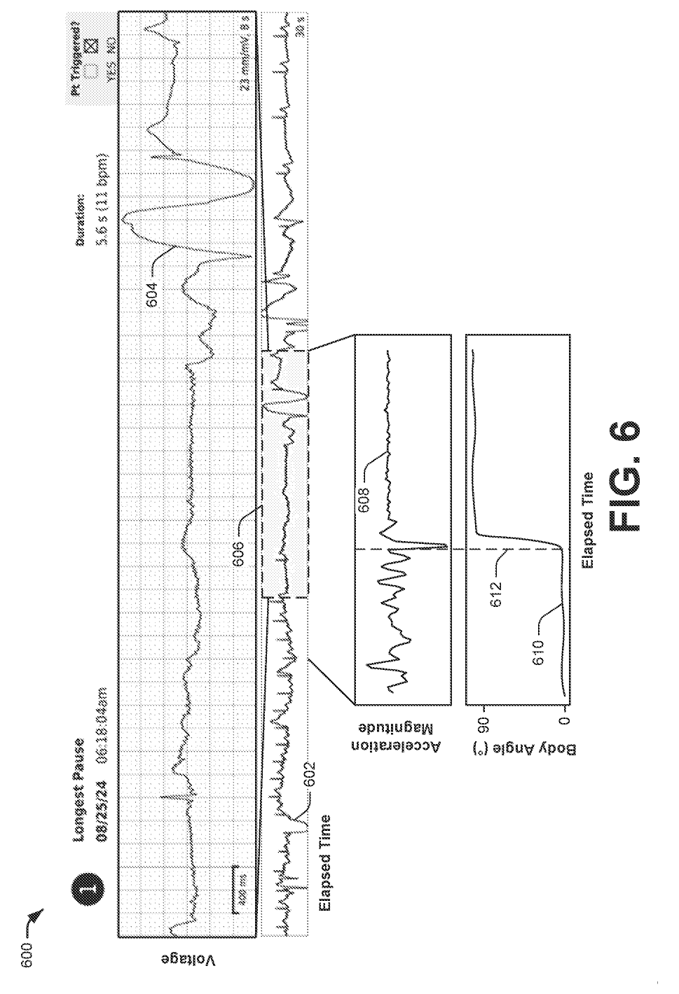

FIG. 6 shows an example 600 of fall detection using chest accelerometer data, demonstrating how the fall detection system 118 may combine multiple measurements of the monitoring device 104 to identify potential fall events. The example 600 includes a voltage plot 602 that displays electrical potential measurements captured over an elapsed time period, providing a measurement of cardiac activity obtained by the monitoring device 104 during the monitoring period. A magnified voltage plot 604 presents a detailed view of voltage variations within a specific time window corresponding to a potential cardiac pause event, indicated as a suspected pause region 606. The suspected pause region 606 corresponds to a region where cardiac electrical activity appears to be interrupted or significantly altered. In one or more implementations, the suspected pause region 606 may be indicated via the cardiac rhythm classification 126 output by the prediction system 114.

However, because there is noise toward the end of the pause, it may be difficult for a clinician to discern whether it was a true pause, a pause resulting in a fall, or the duration of the pause (if a true pause). Additional context of a fall being detected could inform the clinician without relying on patient memory.

As further shown in FIG. 6, an acceleration magnitude plot 608 displays changes in acceleration measurements during an overlapping time period as the suspected pause region 606, beginning before the suspected pause region 606 in this example. The acceleration magnitude plot 608 enables direct temporal correlation between cardiac events and movement patterns. The acceleration magnitude plot 608 may capture various types of movement including normal daily activities, sudden movements, and potential fall events.

The example 600 also includes a body angle plot 610 that tracks the orientation of the monitoring device 104 relative to gravitational forces measured by the accelerometer, providing information about the position and posture of the person 102 during this overlapping time period. The body angle plot 610 may display angle measurements ranging from approximately 0 to 90 degrees, where lower values may indicate more upright positions and higher values may indicate more reclined or horizontal positions.

A fall event 612 may be identified based on the acceleration magnitude plot 608 alone or in combination with the body angle plot 610. By way of example, the acceleration magnitude plot 608 may indicate a sudden freefall followed by impact and then lack of movement, and the body angle plot 610 may indicate a corresponding sudden change in body orientation, such as a rapid transition from a more upright position to a more horizontal position.

The fall detection system 118 may analyze multiple parameters simultaneously to differentiate between genuine fall events and other activities that might produce similar accelerometer signatures. Impact force analysis may involve examining the magnitude and rate of change in acceleration measurements to identify patterns consistent with falls versus other activities such as sitting down quickly or lying down intentionally. Body orientation changes may be evaluated by monitoring the body angle plot 610 for sudden transitions that exceed predetermined thresholds in both magnitude and rate of change. Post-fall movement patterns may be assessed by analyzing accelerometer data following a suspected fall event to determine whether the person 102 remains in a horizontal position for an extended period or exhibits movement patterns consistent with recovery from a fall.

The correlation between the fall event 612 and the suspected pause region 606 may provide clinical context for healthcare providers. For instance, the example 600 shows a temporal correlation between the suspected pause region 606 in the voltage plot 602 and the fall event 612. In this example, the fall event 612 occurs during the cardiac irregularity identified in the suspected pause region 606. Such correlation may help a clinician determine that the suspected pause region 606 corresponds to a true cardiac pause due to the suspected cardiac pause resulting in a fall (e.g., due to dizziness, lightheadedness, and/or fainting during the cardiac pause) without relying on patient memory.

In one or more implementations, when the fall detection system 118 processes at least a portion of the measurements 108 during the observation period and detects a fall event 612 in conjunction with a cardiac irregularity such as the suspected pause region 606, the prediction system 114 may generate an automated alert, an example of which is the notification 134 depicted in FIG. 1. The notification 134 may include detailed information about the timing and characteristics of both the fall event 612 and the associated cardiac irregularity and may be transmitted to person 102, the healthcare provider 132, and/or an emergency contact of the person 102, for example. The notification 134 may provide an alert of a potential medical event that may benefit from medical attention or follow-up evaluation.

In at least one implementation, the notification 134 may not be a real-time notification. For instance, there may be a delay (e.g., seconds, minutes, hours, days) between obtaining the measurements 108 and analyzing the measurements 108 by the fall detection system 118 to detect the fall event 612. In at least one variation, however, the measurements 108 may be at least partially analyzed by the fall detection system 118 substantially at the time of acquisition. Accordingly, the notification 134 may indicate a past/recent fall event 612 during the observation period (e.g., when the notification 134 is delayed) or may indicate a current fall event 612 (e.g., when the notification 134 is real-time).

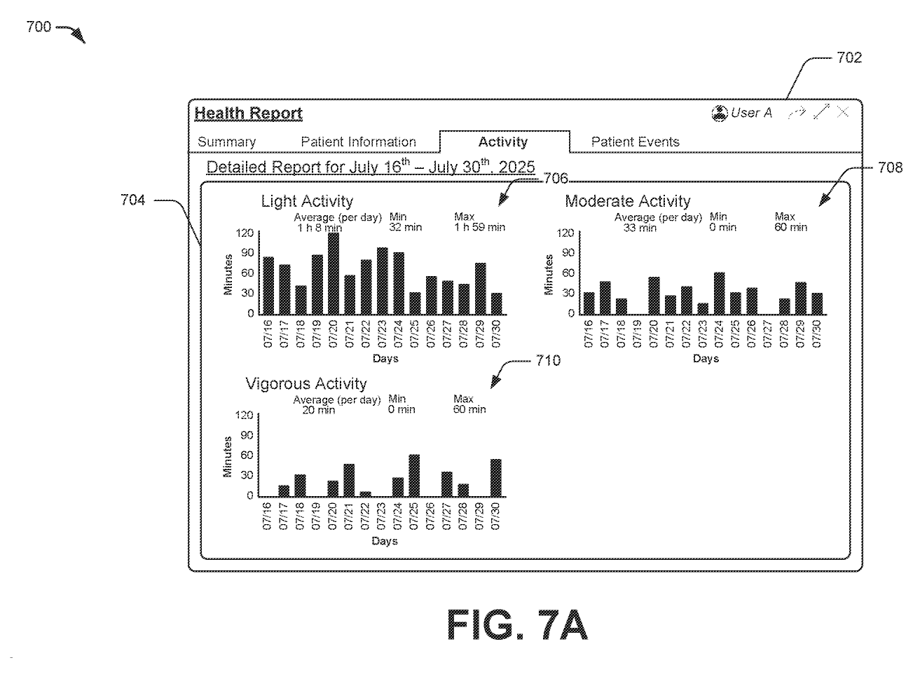

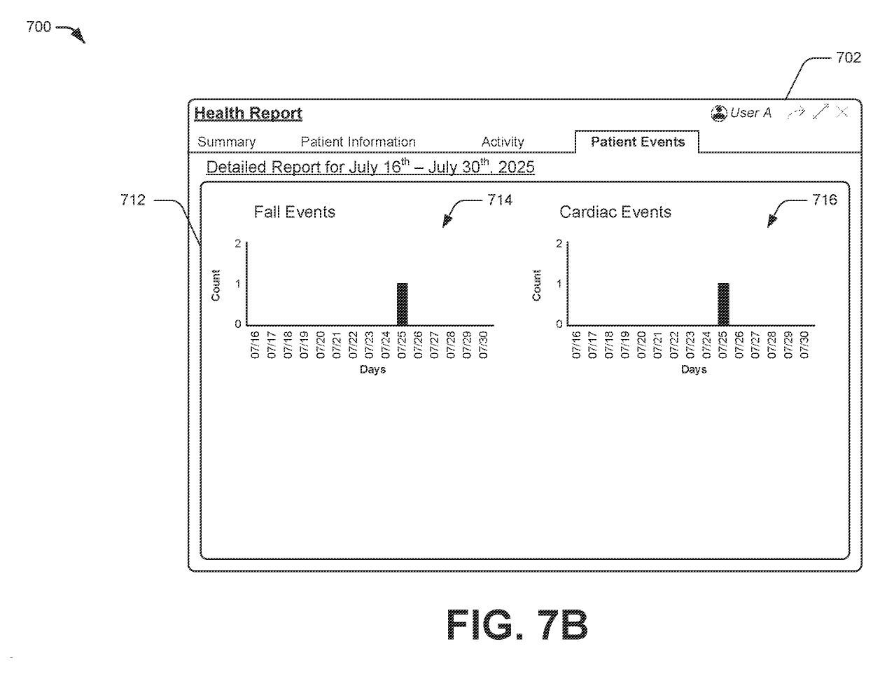

FIGS. 7A and 7B show an example 700 of user interface 702 configurations for displaying health monitoring data and clinical reports. The example 700, for instance, represents one implementation of how activity level and fall detection data collected by the monitoring device 104 during an observation period may be displayed following analysis by the analysis platform 106. The user interface 702 provides a platform for healthcare providers and/or users (e.g., the person 102) to review activity patterns, fall events, and associated cardiac data. The user interface 702 may be implemented on various computing devices, including smartphones, tablets, desktop computers, or dedicated medical workstations.

FIG. 7A depicts the user interface 702 configured to display activity-related health data through an activity report section 704, which may report on detected activity of the person 102 during the observation period. In this example, the activity report section 704 displays activity data for a time period spanning from July 16th to July 30^{th} when the “Activity” tab selected. For example, an input is received at the user interface 702 to select the “Activity” tab, which causes the prediction system 114 to display the activity report section 704.

The activity report section 704 includes multiple graphical representations of activity data that provide visual correlations between different activity intensity levels detected by the accelerometer data 306 and processed by the activity detection system 116, including a light activity graph 706, a moderate activity graph 708, and a vigorous activity graph 710. In the present example, each activity graph displays daily measurements of the corresponding physical activity level over the monitoring period, indicating how much of the corresponding activity was performed per day. While the example 700 shows the activity levels related to the time spent in each activity level, variations are possible. By way of example, the step count, distance traveled, or another type of measurement may be used in addition to or as an alternative to time. Moreover, the activity levels may be classified differently than shown. The light activity graph 706, the moderate activity graph 708, and the vigorous activity graph 710 may provide a visual representation of activity patterns and/or physical activity levels over time and may be further related to other analyses, at least in some examples.

By way of example, the activity graphs in the activity report section 704 may enable healthcare providers to identify periods of increased or decreased activity that may correlate with health events or changes in the condition of the person 102. For example, a notable decrease in moderate or vigorous activity levels may indicate declining health status, while consistent activity patterns may suggest stable physical condition. As described, e.g., with respect to FIG. 3, the prediction system 114 may process the accelerometer data 306 to generate these activity classifications using machine learning models trained on labeled activity data, enabling accurate differentiation between different activity intensities.

FIG. 7B shows the user interface 702 configured to display fall detection and cardiac event correlation data through a patient event report section 712. By way of example, an input is received to select a “Patient Events” tab, which causes the prediction system 114 to display the patient event report section 712. In this configuration, the patient event report section 712 presents event-based information for the same observation period as the activity report section 704. The patient event report section 712 may enable users to analyze relationships between fall events and cardiac irregularities, which may be further correlated with the activity levels of the activity report section 704. This correlation capability addresses clinical scenarios where fall events may be associated with cardiac episodes, providing context for diagnosis and treatment decisions.

The patient event report section 712 includes a fall events graph 714 that displays the occurrence (e.g., count) and timing (e.g., per day in this example) of detected fall events and a cardiac events graph 716 that displays concurrent cardiac event data. In at least one implementation, the user interface 702 may display more granular information in response to the user selecting a particular date, a particular event, etc. in order to present temporal correlations between fall occurrences and cardiac irregularities, such as arrhythmias, pauses, or other cardiac rhythm abnormalities detected through analysis of the electrical data 304 collected by the monitoring device 104.

For instance, user selection of the date (e.g., “07/25”) may cause the user interface 702 to output a visual similar to that shown in the example 600 of FIG. 6, showing a visual correlation between the fall prediction 124 and the cardiac rhythm classification 126. Accordingly, by leveraging accelerometer data and electrical potential data to detect both fall and cardiac events, the prediction system 114 may provide enhanced insights into the complex interplay between movement patterns and cardiovascular activity during daily activities.

Such insights are not possible using conventional techniques that rely on separate analysis of fall detection, activity detection, and cardiac monitoring. In some examples, this further conserves computational resources that would otherwise be expended processing input data types from multiple distinct monitoring devices and/or analysis platforms. Accordingly, by generating multi-modality insights based on correlated accelerometer and cardiac data, the techniques described herein improve operations of devices that implement the prediction system 114.

In one or more implementations, the user interface 702 may generate a comprehensive report that includes timelines of detected fall events, associated cardiac data for each fall event, and recommendations for fall prevention based on the activity graphs of the activity report section 704. The report may combine information from the light activity graph 706, the moderate activity graph 708, the vigorous activity graph 710, the fall events graph 714, and the cardiac events graph 716 to provide users with detailed insights into patient health. In one or more implementations, as a part of the comprehensive report, the user interface 702 may display the step count 120, the cardiac rhythm classification 126, and/or the wellness prediction 128. Accordingly, the user interface 702 may present a multifaceted view of the activity, fall, and cardiac data as well as other physiological observations. This comprehensive presentation may facilitate interpretation of complex movement and cardiac data, potentially leading to earlier detection of fall risk factors and more effective management of related health issues.

The following section describes example procedures for activity level and fall detection using accelerometer data in one or more implementations. Aspects of each of the procedures can be implemented in hardware, firmware, software, or a combination thereof. The procedures are shown as a set of blocks that specify operations that can be performed by one or more devices and are not necessarily limited to the orders shown for performing the operations by the respective blocks. One or more blocks of the procedures, for instance, specify operations that can be programmable by hardware (e.g., processor, microprocessor, controller, firmware) as instructions thereby creating a special purpose machine for carrying out an algorithm as illustrated by the flow diagram. As a result, the instructions are storable on a computer-readable storage medium that causes the hardware to perform the algorithm. In portions of the following discussion, reference will be made to FIGS. 1-6, 7A, and 7B.

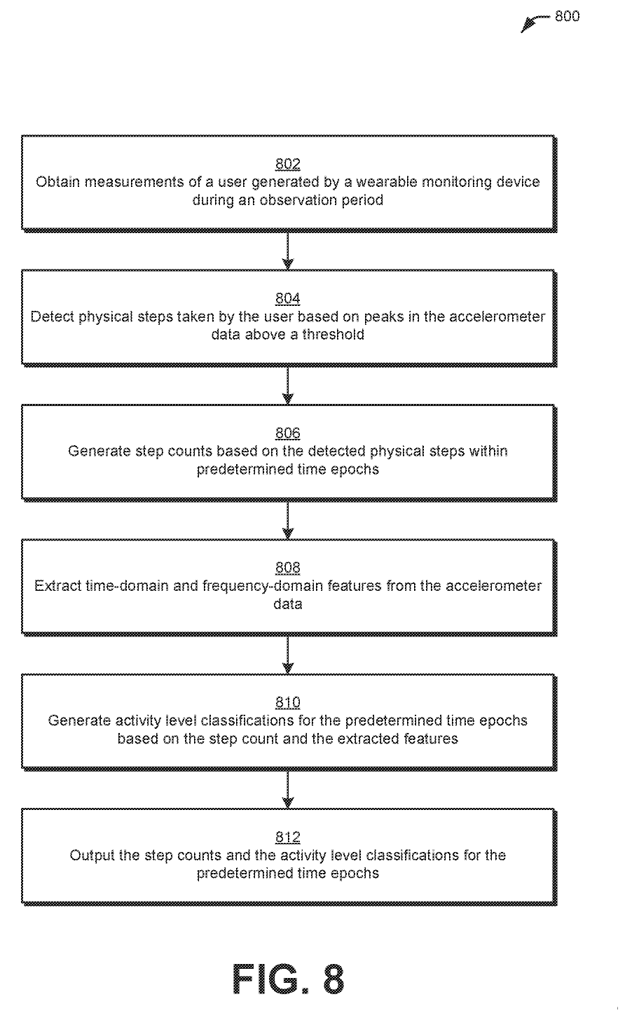

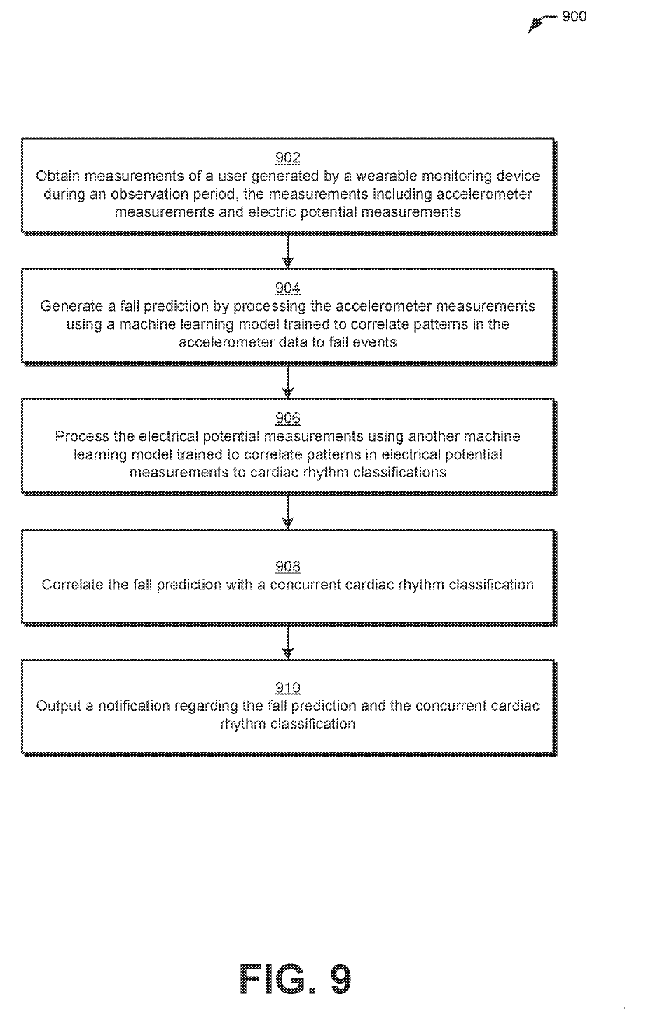

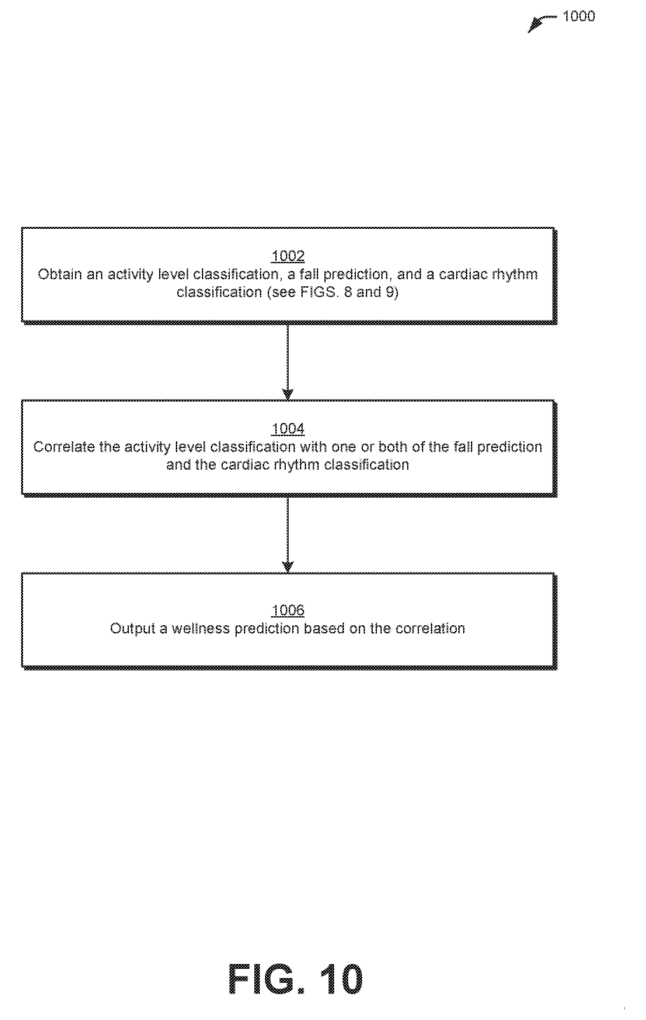

FIG. 8 shows a flow diagram depicting an algorithm as a step-by-step procedure 800 in an example implementation that is performable by a processing device to generate activity level and step count predictions based on accelerometer measurements. The procedure 800 may be performed by the prediction system 114 or other components of the analysis platform 106 to analyze movement patterns and classify physical activity levels of the person 102.

To begin in this example, measurements of a user generated by a wearable monitoring device during an observation period are obtained (block 802). By way of example, the measurements 108 may include the accelerometer data 306, which may be sampled at rate of less than 5 Hz (e.g., 1.6 Hz). In various examples, the monitoring device 104 detects movement and orientation changes of the person 102 using the one or more sensors 202 (e.g., an accelerometer) and produces the accelerometer data 306 based on the detected motion. The monitoring device 104 may at least partially process the accelerometer data 306 locally, before transmission to the analysis platform 106. Alternatively, the analysis platform 106 may be included as part of the monitoring device 104. In yet another example, the monitoring device 104 may transmit raw accelerometer data 306 to the analysis platform 106 for processing. Additionally or alternatively, the monitoring device 104 may compress the sensor data using various data compression techniques to reduce battery usage during transmission (e.g., wireless or wired transmission) to external computing devices.

Physical steps taken by the user are detected based on peaks in the accelerometer data above a threshold (block 804). The activity detection system 116, for instance, analyzes the accelerometer data 306 to identify characteristic patterns that correspond to individual steps taken by the person 102. The step detection process may include examining the acceleration magnitude 402 and comparing the measured values against the threshold 404 to identify step peaks 406 that exceed the predetermined threshold level. The threshold 404 may be dynamically adjusted based on variations in the accelerometer data 306 to account for different walking speeds, terrains, and/or individual movement patterns of the person 102. Alternatively, the threshold 404 may be a fixed value.

Step counts are generated based on the detected physical steps within predetermined time epochs (block 806). The activity detection system 116 may organize the detected step peaks 406 into discrete time periods or epochs to facilitate step counting. By way of example, each epoch may represent a specific duration, such as one minute, five minutes, one hour, one day, or another suitable time interval during which the total number of detected steps is calculated and recorded. The step count 120 may be stored in the storage device 112 for subsequent analysis and correlation with other physiological measurements and/or output for display, e.g., via the accessory device 130.

Time-domain and frequency-domain features are extracted from the accelerometer data (block 808). By way of example, the feature extraction module 318 processes the accelerometer data 306 to identify various characteristics that may be indicative of different types of physical activity. Time-domain features may include statistical measures such as mean, variance, and standard deviation of the acceleration signals, while frequency-domain features may include spectral analysis to identify dominant frequencies and patterns in the movement data. These data features 320 may provide additional information beyond the step count 120 that may be used to distinguish between different activity intensities and movement types.

Activity level classifications are generated for the predetermined time epochs based on the step count and the extracted features (block 810). By way of example, the trained machine learning model 326 receives the step count 120 and the data features 320 as input to classify the activity level of the person 102 during each epoch. The activity level classification 122 may categorize the physical activity into discrete intensity levels, such as sedentary, light, moderate, or vigorous based on the combination of step count and feature analysis. The machine learning model 316 may be trained using historical accelerometer data and corresponding activity classifications to accurately distinguish between different intensity levels of physical activity. In at least one variation, the step count 120 within the epoch is compared to one or more thresholds to classify the activity level and output the activity level classification 122.

The step counts and the activity level classifications are output for the predetermined time epochs (block 812). By way of example, the prediction system 114 may cause display of the step count 120 and/or the activity level classification 122 via the user interface 702 or may incorporate the predictions into another type of health report for review by the healthcare providers 132 and/or the person 102. The output may include detailed graphs and visualizations showing activity patterns over time, such as by displaying activity minutes per day as the light activity graph 706, the moderate activity graph 708, and the vigorous activity graph 710. In various examples, the notification 134 or another type of alert related to the activity level classification 122 may be transmitted to computing devices associated with the person 102 and/or the healthcare providers 132 to facilitate monitoring and assessment of physical activity levels, alone or in combination with other physiological measurements obtained by the monitoring device 104.

FIG. 9 depicts a flow diagram depicting an algorithm as a step-by-step procedure 900 in an example implementation that is performable by a processing device to generate fall predictions correlated with cardiac rhythm classifications. The procedure 900 integrates the accelerometer data 306 and the electrical data 304 obtained from the person 102 by the monitoring device 104 during the observation period to provide comprehensive analysis of fall events and their relationship to cardiac activity.

To begin in this example, measurements of a user generated by a wearable monitoring device during an observation period are obtained, the measurements including accelerometer measurements and electrical potential measurements (block 902). The measurements 108, for instance, are produced by the monitoring device 104 during continuous wear by the person 102. In various examples, the monitoring device 104 detects movement patterns based on the accelerometer data 306 while simultaneously capturing electrical activity of the heart using the one or more sensors 202. The accelerometer data 306 may be sampled at frequencies lower than conventional activity trackers, which typically operate at 25-100 Hz, thereby conserving battery life and memory resources of the monitoring device 104. The electrical data 304 (e.g., electrical potential measurements) may be collected substantially continuously to provide cardiac rhythm information that can be correlated with detected movement patterns.

A fall prediction is generated by processing the accelerometer measurements using a machine learning model trained to correlate patterns in the accelerometer data to fall events (block 904). The machine learning model 316, for instance, is trained using historical accelerometer measurements and labeled fall event data from a user population to perform a fall detection task. The fall detection task may include analyzing impact force, body orientation changes, and post-fall movement patterns to differentiate between genuine falls and other activities with similar accelerometer signatures. For example, the trained machine learning model 326 receives the accelerometer data 306 as input and generates the fall prediction 124 based on identified patterns indicative of fall events. The machine learning model 316 may analyze features such as sudden changes in acceleration magnitude, duration of impact events, and subsequent movement patterns to distinguish falls from activities like sitting down or lying down voluntarily. The machine learning model 316 may further take into account position information determined based on the accelerometer data 306.

The electrical potential measurements are processed using another machine learning model trained to correlate patterns in electrical potential measurements to cardiac rhythm classifications (block 906). The trained machine learning model 326 for cardiac analysis, for instance, is trained using historical electrical potential measurements and corresponding cardiac rhythm labels to identify various arrhythmias and cardiac events. In some cases, the trained machine learning model 326 processes the electrical data 304 to generate the cardiac rhythm classification 126, which may include identification of conditions such as atrial fibrillation, pauses, or other cardiac irregularities. The trained machine learning model 326 for cardiac analysis may examine features such as heart rate variability, rhythm patterns, and electrical signal morphology to classify different types of cardiac events that may occur before, during, or after fall incidents.

The fall prediction is correlated with a concurrent cardiac rhythm classification (block 908). The fall detection system 118, for instance, performs temporal analysis to identify relationships between the fall prediction 124 and the cardiac rhythm classification 126 that occur within a specified time window. In various examples, the correlation process may identify cardiac events that precede fall incidents by seconds or minutes, suggesting a causal relationship where cardiac irregularities may contribute to fall risk. The fall detection system 118 may examine the timing of the fall prediction 124 relative to the cardiac rhythm classification 126 to determine whether cardiac events such as pauses or arrhythmias coincide with or immediately precede detected falls. This correlation analysis may provide clinical insights for healthcare providers regarding the underlying causes of fall events and potential cardiac-related fall risks.

A notification regarding the fall prediction and the concurrent cardiac rhythm classification is output (block 910). For example, the prediction system 114 causes generation of the notification 134 that includes information about both the detected fall event and any associated cardiac irregularities. The notification 134 may be transmitted to the healthcare provider 132 or displayed via a user interface associated with the analysis platform 106, e.g., on the accessory device 130. In various examples, the notification 134 may include detailed timing information, severity assessments, and recommendations for further evaluation when fall events are detected in conjunction with cardiac irregularities. The notification 134 may also be incorporated into comprehensive reports that provide healthcare providers with context for clinical decision-making without relying on patient memory of events, particularly in cases where cardiac pauses or other irregularities may have contributed to fall incidents.

FIG. 10 depicts a flow diagram depicting an algorithm as a step-by-step procedure 1000 in an example implementation that is performable by a processing device to generate wellness predictions. The procedure 1000 represents an integrated approach to wellness analysis that combines multiple physiological measurements and predictions to generate comprehensive health assessments. In some cases, the procedure 1000 may be implemented by the prediction system 114 to correlate various outputs of the one or more predictions 110 and provide healthcare providers with a holistic view of patient wellness.

An activity level classification, a fall prediction, and a cardiac rhythm classification are obtained (block 1002). By way of example, the activity level classification 122, the fall prediction 124, and cardiac rhythm classification 126 may be generated using the procedures described in connection with FIGS. 8 and 9. In various implementations, the one or more predictions 110, including the activity level classification 122, the fall prediction 124, and the cardiac rhythm classification 126 are generated during or after the observation period of the monitoring device 104.

The activity level classification is correlated with one or both of the fall prediction and the cardiac rhythm classification (block 1004). By way of example, the analysis platform 106 may perform a temporal analysis to identify relationships between different physiological events (e.g., cardiac rhythm abnormalities and/or fall events) and activity states. For instance, the analysis platform 106 may determine whether fall events occur more frequently during specific activity levels or in conjunction with particular cardiac rhythm abnormalities. The correlation may also examine patterns where cardiac irregularities precede changes in activity levels or fall incidents. In one or more implementations, the analysis platform 106 may identify sedentary periods that coincide with arrhythmic episodes, or vigorous activity periods that trigger specific cardiac responses.

A wellness prediction is output based on the correlation (block 1006). By way of example, the wellness prediction 128 may represent a comprehensive assessment that integrates multiple health indicators to provide actionable insights for healthcare providers 132 and/or the person 102. For example, the wellness prediction 128 may indicate increased fall risk during periods of cardiac irregularity or may suggest modifications to activity levels based on observed correlations between exercise intensity and arrhythmic events. The wellness prediction 128 may be formatted as part of a comprehensive health report that includes recommendations for lifestyle modifications, medical interventions, or monitoring adjustments, which may be output via the user interface 702, for example, and/or via the accessory device 130.

The analysis platform 106 may employ advanced signal processing techniques to synchronize data from multiple sensors and ensure accurate temporal correlation of events. Machine learning models within the prediction system 114 (e.g., the trained machine learning model 326) may be trained on multi-modal datasets that include concurrent accelerometer, cardiac, and other physiological measurements to improve the accuracy of wellness predictions. The storage device 112 may maintain historical data across multiple observation periods to enable longitudinal wellness trend analysis and personalized health insights. In various examples, the wellness prediction 128 may incorporate demographic factors, medical history, and environmental conditions to provide contextually relevant health assessments that support clinical decision-making and patient care optimization.

The integrated approach represented by the procedure 1000 enables the analysis platform 106 to provide multi-modal health insights that would not be achievable through analysis of individual physiological parameters alone. The monitoring device 104 may be configured with various combinations of the one or more sensors 202 to support this comprehensive analysis approach (e.g., accelerometer sensors, electrocardiogram electrodes, pulse oximetry sensors, and so forth) to capture diverse physiological signals.

The various functional units illustrated in the figures and/or described herein are implemented in any of a variety of different manners such as hardware circuitry, software or firmware executing on a programmable processor, or any combination of two or more of hardware, software, and firmware. The methods provided are implemented in any of a variety of devices, such as a general-purpose computer, a processor, or a processor core. Suitable processors include, by way of example, a general purpose processor, a special purpose processor, a conventional processor, a digital signal processor (DSP), a graphics processing unit (GPU), a parallel accelerated processor, a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine.

In one or more implementations, the methods and procedures provided herein are implemented in a computer program, software, or firmware incorporated in a non-transitory computer-readable storage medium for execution by a general-purpose computer or a processor. Examples of non-transitory computer-readable storage mediums include a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

It should be understood that many variations are possible based on the disclosure herein. Although features and elements are described above in particular combinations, each feature or element is usable alone without the other features and elements or in various combinations with or without other features and elements.

What is claimed is:

1. A method implemented by a processing device, the method comprising:
obtaining measurements of a user generated by a wearable monitoring device during an observation period, the measurements including accelerometer data;
detecting physical steps taken by the user based on peaks in the accelerometer data above a threshold;
generating a step count based on the detected physical steps within predetermined time epochs;
generating an activity level classification of the user for the predetermined time epochs based on the step count within a corresponding predetermined time epoch; and
outputting the step count and the activity level classification for the predetermined time epochs.

2. The method of claim 1, wherein the accelerometer data are collected at a sampling rate of less than 5 Hertz.

3. The method of claim 1, wherein the activity level classification includes categorizing an activity of the user during the predetermined time epochs as one of sedentary, light, moderate, or vigorous based on the step count within the predetermined time epochs.

4. The method of claim 3, further comprising extracting time-domain and frequency-domain features from the accelerometer data, and wherein generating the activity level classification is additionally based on the extracted features.

5. The method of claim 1, further comprising:
generating a fall prediction by processing the accelerometer data using a machine learning model trained to correlate patterns in the accelerometer data to fall events; and
outputting the fall prediction.

6. The method of claim 5, further comprising training the machine learning model to perform the fall prediction using historical accelerometer data and historical outcome data of a user population as training data, wherein the historical accelerometer data is sampled at a same sampling rate as the accelerometer data obtained by the wearable monitoring device.

7. The method of claim 5, wherein the measurements further include electrical potential measurements of a heart of the user, and the method further comprises:
generating a cardiac rhythm classification by processing the electrical potential measurements using another machine learning model trained to correlate patterns in the electrical potential measurements to cardiac rhythm classifications.

8. The method of claim 7, wherein the cardiac rhythm classification includes an indication of an arrhythmia, and the method further comprises:
outputting a notification in response to the fall prediction temporally correlating with the arrhythmia.

9. The method of claim 7, wherein the cardiac rhythm classification is one of atrial fibrillation, bradycardia, ventricular arrhythmia, heart block, premature ventricular contraction, supraventricular tachycardia, or normal sinus rhythm.

10. A processing device, comprising:
one or more processors; and
memory having stored computer-readable instructions that are executable by the one or more processors to perform operations comprising:
obtaining measurements of a user generated by a wearable monitoring device during an observation period, the measurements including accelerometer data;
detecting physical steps taken by the user based on peaks in the accelerometer data above a threshold;
generating a step count based on the detected physical steps within predetermined time epochs;
generating an activity level classification of the user for the predetermined time epochs based on the step count within a corresponding predetermined time epoch; and
outputting at least one of the step count or the activity level classification for the predetermined time epochs in a user interface.

11. The processing device of claim 10, wherein the accelerometer data are obtained by an accelerometer of the wearable monitoring device at a sampling rate of less than 5 Hertz.

12. The processing device of claim 10, wherein the activity level classification includes categorizing an activity of the user during the predetermined time epochs as one of sedentary, light, moderate, or vigorous based on the step count within the predetermined time epochs.

13. The processing device of claim 10, wherein the operations further comprise generating a fall prediction by processing the accelerometer data using a machine learning model trained to correlate patterns in the accelerometer data to fall events.

14. The processing device of claim 13, wherein the measurements further include electrical potential measurements of a heart of the user, and the operations further comprise:
generating a cardiac rhythm classification by processing the electrical potential measurements using another machine learning model trained to correlate patterns in the electrical potential measurements to cardiac rhythm classifications; and
outputting the cardiac rhythm classification in the user interface.

15. The processing device of claim 14, wherein the operations further comprise:
correlating the activity level classification with one or both of the fall prediction and the cardiac rhythm classification; and
outputting a wellness prediction based on the correlating.

16. A system, comprising:
a wearable monitoring device that is wearable by a user to detect one or more measurements of the user during an observation period, the one or more measurements including accelerometer measurements and electrical potential measurements of a heart of the user; and
a computing device configured to:
receive the one or more measurements from the wearable monitoring device;
generate activity level classifications of the user within predetermined time epochs based on the accelerometer measurements within a corresponding predetermined time epoch;
generate a fall prediction by processing the accelerometer measurements using a machine learning model trained to correlate patterns in the accelerometer measurements to fall events; and
output the activity level classifications and the fall prediction.

17. The system of claim 16, wherein the accelerometer measurements are collected at a sampling rate of less than 5 Hertz.

18. The system of claim 16, wherein the computing device is further configured to generate a cardiac rhythm classification by processing the electrical potential measurements using another machine learning model trained to correlate patterns in the electrical potential measurements to cardiac rhythm classifications.

19. The system of claim 18, wherein the computing device is further configured to:
correlate the fall prediction with a concurrent cardiac rhythm classification; and
output a notification regarding the fall prediction and the concurrent cardiac rhythm classification.

20. The system of claim 18, wherein to generate the activity level classifications, the computing device is further configured to:
detect steps the user has taken based on peaks in the accelerometer measurements that exceed a threshold;
generate step counts within the predetermined time epochs based on the detected steps within a given predetermined time epoch; and
indicate a given activity level classification for the given predetermined time epoch as one of sedentary, light, moderate, or vigorous based on the step counts within the given predetermined time epoch.
